(19) United States
(12) Patent Application Publication
(10) Pub. No.: US 2023/0355865 A1
(43) Pub. Date: Nov. 9, 2023

(54) INFUSION SITE RETAINER FOR MAINTAINING INFUSION TUBING

(71) Applicant: MedicaMetrix, Inc., Lowell, MA (US)

(72) Inventors: Daniel E. Larkin, St. Paul, MN (US); Matt Leyden, St. Paul, MN (US); Matthew Goulet, St. Paul, MN (US); Jacob Stoerz, St. Paul, MN (US)

(21) Appl. No.: 18/025,718

(22) PCT Filed: Sep. 13, 2021
(86) PCT No.: PCT/US2021/050062

§ 371 Date: Mar. 10, 2023

Related U.S. Application Data
(60) Provisional application No. 63/146,947, filed on Feb. 8, 2021.
(60) Provisional application No. 63/116,524, filed on Nov. 20, 2020.
(60) Provisional application No. 63/077,131, filed on Sep. 11, 2020.

(51) Int. Cl. A61M 5/14

(52) U.S. Cl. CPC A61M 5/1418; A61M 2005/1416

(57) ABSTRACT

An infusion site retainer configured to maintain tubing coupled to an infusion port includes a base configured to be secured to a patient and comprises a patient surface opposite a first surface and comprises a sidewall surface substantially perpendicular to the patient surface and the first surface. At least one trench is formed in the first surface of the base to secure the tubing to the base. At least one trench is formed in the sidewall surface of the base to secure the tubing to the base.

### FIELD OF INVENTION

The following disclosure relates to the medical field and more particularly to medical devices.

### BACKGROUND OF THE INVENTION

Intravenous infusion is the introduction of a liquid, such as a saline solution, into a vein of a patient for the therapeutic treatment of the patient. The intravenous infusion is introduced to a body site through an infusion port that generally has a needle suited for accessing the vein and tubing extending from the infusion port that communicates with a fluid receptacle, such as a sealed plastic bag.

Intravenous infusion is a common medical practice that is done in outpatient procedures, inpatient and other surgical procedures, and long-term care procedures. Some patients are sedated and move in an involuntary manner that has the potential to disrupt or remove the infusion port from the infusion site. Other patients are uncomfortable with the notion of intravenous infusion and are fearful that their voluntary movements might displace the infusion port. Still other patients are confused or agitated and pull at the tubing connected to the infusion port.

For therapeutic and other reasons, it is desirable to provide improved healthcare outcomes for patients undergoing intravenous infusion.

### SUMMARY OF THE INVENTION

One embodiment is directed to an infusion site retainer configured to maintain tubing coupled to an infusion port. The infusion site retainer includes a base configured to be secured to a patient and comprises a patient surface opposite a first surface and comprises a sidewall surface substantially perpendicular to the patient surface and the first surface. At least one trench is formed in the first surface of the base to secure the tubing to the base. At least one trench is formed in the sidewall surface of the base to secure the tubing to the base.

### BRIEF DESCRIPTION OF THE DRAWINGS

FIG. 1 is a diagram illustrating a first perspective view of an infusion site retainer according to one embodiment.

FIG. 2 is a diagram illustrating a second perspective view of the infusion site retainer shown in FIG. 1 according to one embodiment.

FIG. 3 is a diagram illustrating a third perspective view of the infusion site retainer shown in FIG. 1 according to one embodiment.

FIG. 4 is a diagram illustrating a top view of the infusion site retainer shown in FIG. 1 attached to a patient's arm according to one embodiment.

FIG. 5 is a diagram illustrating a top view of an infusion site retainer attached to a patient's arm according to another embodiment.

FIG. 6A is a diagram illustrating an assembly view of an infusion site retainer according to another embodiment.

FIG. 6B is a diagram illustrating a top view of the infusion site retainer shown in FIG. 6A after assembly according to one embodiment.

FIG. 6C is a diagram illustrating a side view of the infusion site retainer shown in FIG. 6A after assembly according to one embodiment.

FIG. 7 is a diagram illustrating an infusion site retainer according to another embodiment.

FIGS. 8 is a diagram illustrating an infusion site retainer according to another embodiment.

FIG. 9 is a diagram illustrating dimensions of an infusion site retainer according to an embodiment.

FIG. 10 is a diagram illustrating an infusion site retainer according to additional embodiments.

FIG. 11 is a diagram illustrating an infusion site retainer according to another embodiment.

FIG. 12 is a diagram illustrating dimensions of an infusion site retainer according to an embodiment.

FIG. 13 is a diagram illustrating an infusion site retainer according to additional embodiments.

FIG. 14 is a diagram illustrating an infusion site retainer according to another embodiment.

FIG. 15 is a diagram illustrating an infusion site retainer according to additional embodiments.

FIG. 16 is a diagram illustrating an infusion site retainer according to another embodiment.

### DETAILED DESCRIPTION OF THE INVENTION

Features of the various examples and embodiments disclosed herein may be combined.

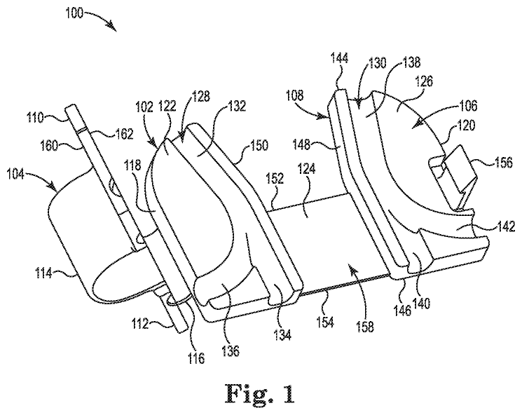

FIG. 1 is a diagram illustrating a first perspective view of an infusion site retainer 100 according to one embodiment. Infusion site retainer 100 includes a base 102 and a cover 104. Base 102 is attachable to a patient. In one embodiment, infusion site retainer 100 is provided as a stand-alone component that is attached to the limb of the patient in any manner deemed suitable by the healthcare provider.

Base 102 includes a first surface or top surface 106 opposite a patient surface or bottom surface 108. In one embodiment, patient surface 108 includes an adhesive configured to attach base 102 to the patient. In one form of this embodiment, the adhesive is initially covered by a cover layer that can be easily peeled off when the retainer 100 is about to be applied to a patient. Base 102 includes opposing first and second lateral sides 118, 120, and a proximal end 146 opposite a distal end 144.

Base 102 includes first lateral base portion 122, central base portion 124, and second lateral base portion 126. Central base portion 124 is positioned between first lateral base portion 122 and second lateral base portion 126. Central base portion 124 extends between an inner sidewall 150 of first lateral base portion 122 and an inner sidewall 148 of second lateral base portion 126. First lateral base portion 122 and second lateral base portion 126 have a larger height (thickness) than central base portion 124 such that the sidewalls 148 and 150 and the top surface 106 of central base portion 124 define a channel 158. The channel 158 extends from a proximal end 154 of central base portion 124 to a distal end 152 of central base portion 124.

In one embodiment, base 102 has a length extending between ends 144, 146 of approximately 3.5 cm and a width extending between sides 118, 120 of about 3.5 cm. In another embodiment, base 102 has a length extending between ends 144, 146 of approximately 2 cm and a width extending between sides 118, 120 of about 5 cm. In yet another embodiment, base 102 has a length extending between ends 144, 146 of approximately 5 cm and a width extending between sides 118, 120 of about 6 cm. In one form of this embodiment, each of base portions 122, 124, and 126 has a width of about 2 cm, and base portions 122 and 126 have a length of about 5 cm, and base portion 124 has length of about 3 cm. In one embodiment, base portions 122 and 126 have a height or thickness of about 4 mm, and base portion 124 has a height or thickness of about 0.5 mm. Other embodiments may have different dimensions.

First lateral base portion 122 includes a trench 128 formed in the first surface 106 of the base portion 122. Trench 128 includes a first a first trench portion 132 that extends from the distal end 144 of the base 102 toward the proximal end 146 of the base 102. First trench portion 132 branches into a second trench portion 134 and a third trench portion 136. In one embodiment, second trench portion 134 is substantially parallel to first trench portion 132 or is slightly angled with respect to first trench portion 132. Second trench portion 134 extends from first trench portion 132 to the proximal end 146 of the base 102. Third trench portion 136 curves laterally away from first trench portion 132 and is substantially perpendicular to first trench portion 132 and second trench portion 134. Third trench portion 136 extends from first trench portion 132 to the lateral side 118 of the base 102.

Second lateral base portion 126 includes a trench 130 formed in the first surface 106 of the base portion 126. Trench 130 includes a first a first trench portion 138 that extends from the distal end 144 of the base 102 toward the proximal end 146 of the base 102. First trench portion 138 branches into a second trench portion 140 and a third trench portion 142.

In one embodiment, second trench portion 140 is substantially parallel to first trench portion 138 or is slightly angled with respect to first trench portion 138. Second trench portion 140 extends from first trench portion 138 to the proximal end 146 of the base 102. Third trench portion 142 curves laterally away from first trench portion 138 and is substantially perpendicular to first trench portion 138 and second trench portion 140. Third trench portion 142 extends from first trench portion 138 to the lateral side 120 of the base 102.

In one embodiment, base 102 is integrally formed from a recyclable or biodegradable plastic to include trenches 128 and 130. Trench 128 is configured to couple a first portion of the infusion tubing to base 102, and trench 130 is configured to couple a second portion of the infusion tubing to base 102. In one embodiment, trenches 128 and 130 are configured to secure the first and second portions of the tubing in a substantially immobile relationship relative to the base 102, without crimping the tubing. For trench 128, the first portion of the infusion tubing may be secured in the first trench portion 132 and the second trench portion 134 to maintain the first portion of the tubing substantially straight or at a slight angle. Alternatively, the first portion of the infusion tubing may be secured in the first trench portion 132 and the third trench portion 136. Similarly, for trench 130, the second portion of the infusion tubing may be secured in the first trench portion 138 and the second trench portion 140 to maintain the second portion of the tubing substantially straight or at a slight angle. Alternatively, the second portion of the infusion tubing may be secured in the first trench portion 138 and the third trench portion 142.

In one embodiment, trenches 128 and 130 have a semicircular cross-sectional shape, and are sized to enable the infusion tubing to be pressed into the trenches 128 and 130 in a manner that captures the infusion tubing. In one embodiment, the trenches 128 and 130 are configured to clamp down upon or grab the infusion tubing, and in this manner prevent the infusion tubing from tugging on the infusion port.

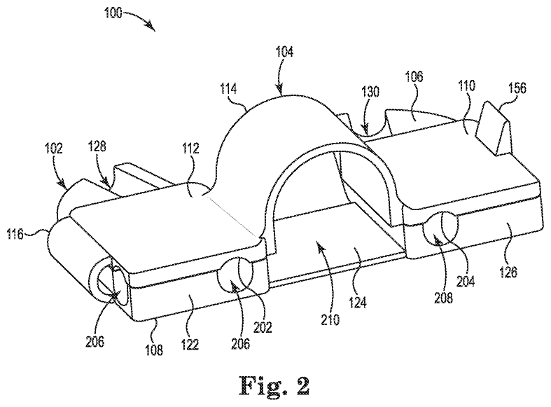

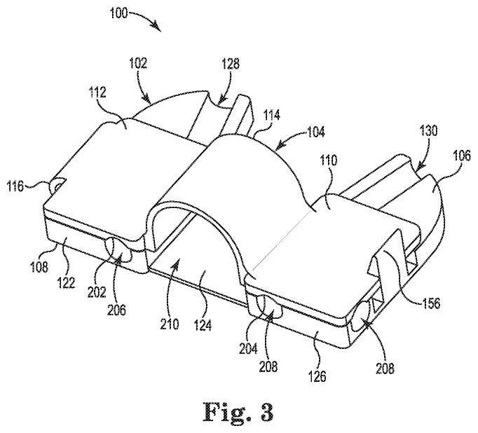

Cover 104 includes a first cover portion 110, a second cover portion 112, and a semi-cylindrical protrusion 114. Protrusion 114 is positioned between first cover portion 110 and second cover portion 112. Cover 104 has a top surface 160 and a bottom surface 162. Cover 104 also includes a hinge attachment feature 116 that pivotally connects the cover 104 to the lateral side 118 of the base 102. Cover 104 is configured to be pivoted via hinge attachment feature 116 between an open position as shown in FIG. 1 and a closed position as shown in FIGS. 2 and 3. Cover 104 may be maintained in the closed position via a clip 156 on the lateral side 120 of the base 102. After closing the cover 104, the clip 156 is snapped over the top surface 160 of the first cover portion 110 to hold the cover 104 in the closed position. The cover 104 is closed after the tubing is inserted into the trenches 128 and 130 to help secure the tubing in the base 102. Other embodiments may not include a cover. In retainers with a cover 104 according to one embodiment, the trenches 128 and 130 are about 3 mm deep to accommodate standard 3 mm diameter tubing, and in retainers without a cover 104 according to one embodiment, the trenches 128 and 130 are about 4 mm deep.

The infusion site retainer 100 is attached to the patient by a healthcare worker in a suitable clean or antiseptic manner before or after the healthcare worker introduces an infusion port to the patient at the infusion site. The infusion port includes a needle that is introduced to a vein (or broadly, to the circulatory system of the patient). Infusion tubing extends from the infusion port to an infusion reservoir, which is typically a plastic bag containing infusion liquid.

FIG. 2 is a diagram illustrating a second perspective view of the infusion site retainer 100 shown in FIG. 1 according to one embodiment. FIG. 3 is a diagram illustrating a third perspective view of the infusion site retainer shown in FIG. 1 according to one embodiment. In FIGS. 2 and 3, cover 104 is in a closed position. In the closed position, cover 104 laterally extends across the entire base 102 from the lateral side 118 to the lateral side 120 (FIG. 1), and longitudinally extends from the proximal end 146 to about the middle of the first trench portions 132 and 138 (FIG. 1).

Trenches 202 and 204 are formed in the bottom surface 162 of the cover 104. Trenches 202 and 204 have a semicircular cross-sectional shape and are shallower than trenches 128 and 130 in the illustrated embodiment. In the closed position, trench 202 is aligned with and lies directly over trench 128, and the combination of trench 202 and trench 128 forms a tunnel 206 with a substantially circular cross section. In the closed position, trench 204 is aligned with and lies directly over trench 130, and the combination of trench 204 and trench 130 forms a tunnel 208 with a substantially circular cross section.

In the closed position, a tunnel 210 is also formed, which is defined by the semi-cylindrical protrusion 114, the central base portion 124, and the sidewalls 148 and 150. The tunnel 210 extends between proximal end 154 of central base portion 124 and distal end 152 of central base portion 124. The tunnel 210 is sized to receive a needle or infusion port.

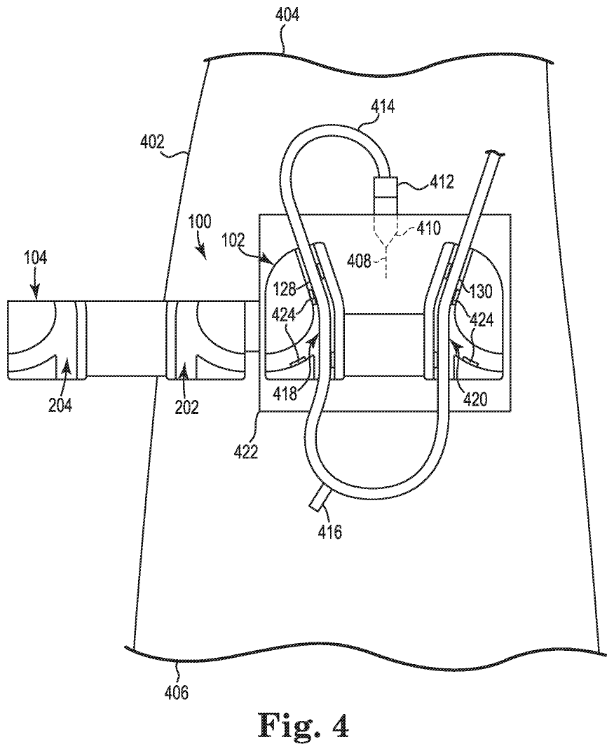

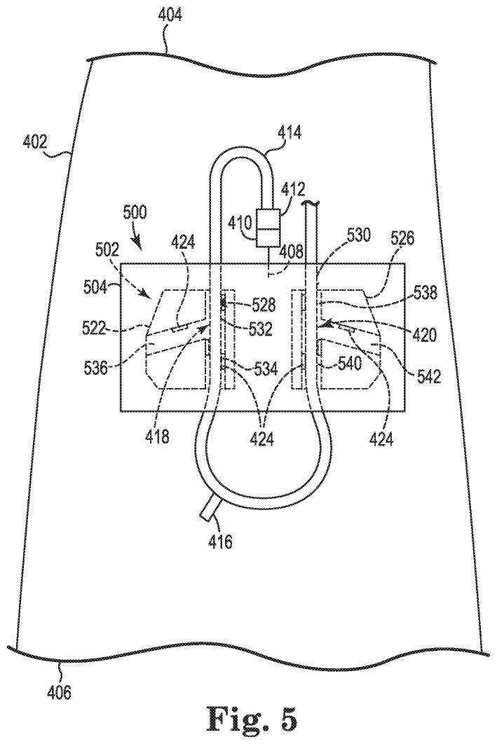

FIG. 4 is a diagram illustrating a top view of the infusion site retainer 100 shown in FIG. 1 attached to a patient's arm 402 according to one embodiment. Arm 402 includes a distal region 404 closer to the patient's hand, and a proximal region 406 farther away from the patient's hand. A needle 408 is inserted into the patient's arm 402 and is coupled to an infusion port 410. Infusion tubing 414 includes a connector 412 configured to be coupled to the infusion port 410. The infusion tubing 414 also includes an infusion port 416 for the introduction of fluids into the patient. The infusion tubing 414 is shown with a smaller diameter in FIGS. 4 and 5 than it would actually appear to allow the infusion tubing elements to be more easily distinguished from the elements of the infusion site retainer 100 and 500.

A film dressing 422 is applied to the arm and covers the needle 408 and a portion of the infusion port 410 (as shown by the dashed lines). In one embodiment, the film dressing 422 is a Tegaderm™ film dressing. The infusion site retainer 100 is adhesively attached to a top surface of the film dressing 422 adjacent to the needle 408.

A first portion 418 of the infusion tubing 414 has been inserted into the trench 128 of the first base portion 122, and a second portion 420 of the infusion tubing 414 has been inserted into the trench 130 of the second base portion 126. More specifically, the first portion 418 of the infusion tubing 414 has been inserted into the first trench portion 132 and the second trench portion 134, and the second portion 420 of the infusion tubing 414 has been inserted into the first trench portion 138 and the second trench portion 140. Alternatively, one or both of the third trench portions 136 and 142 may be used. In the illustrated embodiment, the infusion site retainer 100 includes locking features 424 that help lock the tubing 414 in place in the trenches 128 and 130. Each of the locking features 424 extends over and covers a small portion of one of the trenches 128 and 130. In the illustrated embodiment, four locking features 424 are used for each of the trenches 128 and 130, with the locking features 424 being positioned on alternating sides of the trench along the length of the trench. The cover 104 may be closed over the base 102 to further secure the infusion tubing 414 in place.

FIG. 5 is a diagram illustrating a top view of an infusion site retainer 500 attached to a patient's arm 402 according to another embodiment. Arm 402 includes a distal region 404 closer to the patient's hand, and a proximal region 406 farther away from the patient's hand. A needle 408 is inserted into the patient's arm 402 and is coupled to an infusion port 410. Infusion tubing 414 includes a connector 412 configured to be coupled to the infusion port 410. The infusion tubing 414 also includes an infusion port 416 for the introduction of fluids into the patient.

Infusion site retainer 500 includes a base 502 and a cover 504. Base 502 is attachable to a patient. Base 502 includes a first surface or top surface opposite a patient surface or bottom surface. In one embodiment, the patient surface includes an adhesive configured to attach base 502 to the patient. In one form of this embodiment, the adhesive is covered by a cover layer that can be easily peeled off when the retainer 500 is about to be applied to a patient.

Base 502 includes first lateral base portion 522 and second lateral base portion 526, which are separate and distinct elements in the illustrated embodiment. In one embodiment, base 502 has dimensions that are similar to those of the base 102 described above.

First lateral base portion 522 includes a trench 528 formed in the top surface of the base portion 522. Trench 528 includes a first a first trench portion 532 that extends from the distal end of the base 502 toward the proximal end of the base 502. First trench portion 532 branches into a second trench portion 534 and a third trench portion 536. In the illustrated embodiment, second trench portion 534 is substantially parallel to first trench portion 532. Second trench portion 534 extends from first trench portion 532 to the proximal end of the base 502. Third trench portion 536 branches laterally away from first trench portion 532 and is close to perpendicular to first trench portion 532 and second trench portion 534. Third trench portion 536 extends from first trench portion 532 to the lateral side of the base 502.

Second lateral base portion 526 includes a trench 530 formed in the top surface of the base portion 526. Trench 530 includes a first a first trench portion 538 that extends from the distal end of the base 502 toward the proximal end of the base 502. First trench portion 538 branches into a second trench portion 540 and a third trench portion 542. In the illustrated embodiment, second trench portion 540 is substantially parallel to first trench portion 538. Second trench portion 540 extends from first trench portion 538 to the proximal end of the base 502. Third trench portion 542 branches laterally away from first trench portion 538 and is close to perpendicular to first trench portion 538 and second trench portion 540. Third trench portion 542 extends from first trench portion 538 to the lateral side of the base 502.

In one embodiment, base 502 is integrally formed from a recyclable or biodegradable plastic to include trenches 528 and 530. Trench 528 is configured to couple a first portion of the infusion tubing to base 502, and trench 530 is configured to couple a second portion of the infusion tubing to base 502. In one embodiment, trenches 528 and 530 are configured to secure the first and second portions of the tubing in a substantially immobile relationship relative to the base 502, without crimping the tubing.

For trench 528, the first portion of the infusion tubing may be secured in the first trench portion 532 and the second trench portion 534 to maintain the first portion of the tubing substantially straight or at a slight angle. Alternatively, the first portion of the infusion tubing may be secured in the first trench portion 532 and the third trench portion 536, which forms a larger curve in the infusion tubing. Similarly, for trench 530, the second portion of the infusion tubing may be secured in the first trench portion 538 and the second trench portion 540 to maintain the second portion of the tubing substantially straight or at a slight angle. Alternatively, the second portion of the infusion tubing may be secured in the first trench portion 538 and the third trench portion 542, which forms a larger curve in the infusion tubing.

In one embodiment, trenches 528 and 530 have a semicircular cross-sectional shape, and are sized to enable the infusion tubing to be pressed into the trenches 528 and 530 in a manner that captures the infusion tubing. In one embodiment, the trenches 528 and 530 are configured to clamp down upon or grab the infusion tubing, and in this manner prevent the infusion tubing from tugging on the infusion port.

In the illustrated embodiment, a first portion 418 of the infusion tubing 414 has been inserted into the trench 528 of the first base portion 522, and a second portion 420 of the infusion tubing 414 has been inserted into the trench 530 of the second base portion 526. More specifically, the first portion 418 of the infusion tubing 414 has been inserted into the first trench portion 532 and the second trench portion 534, and the second portion 420 of the infusion tubing 414 has been inserted into the first trench portion 538 and the second trench portion 540. Alternatively, one or both of the third trench portions 536 and 542 may be used. In the illustrated embodiment, the infusion site retainer 500 includes locking features 424 that help lock the tubing 414 in place in the trenches 528 and 530. Each of the locking features 424 extends over and covers a small portion of one of the trenches 528 and 530. In the illustrated embodiment, four locking features 424 are used for each of the trenches 528 and 530, with the locking features 424 being positioned on alternating sides of the trench along the length of the trench.

In one embodiment, cover 504 is very flexible and substantially transparent or translucent, and includes an adhesive surface configured to be attached to the patient's arm 402. In one embodiment, the cover 504 is a Tegaderm™ film dressing. In the illustrated embodiment, cover 504 completely covers the base portions 522 and 526, covers the portions of the infusion tubing inserted into the base portions 522 and 526, and covers the infusion needle 408, so all of these portions are shown with dashed lines. The cover 504 is applied after the tubing is inserted into the trenches 528 and 530 to help secure the tubing in the base 502. Other embodiments may not include a cover.

The infusion site retainer 500 is attached to the patient by a healthcare worker in a suitable clean or antiseptic manner before or after the healthcare worker introduces an infusion port to the patient at the infusion site. The infusion port includes a needle that is introduced to a vein (or broadly, to the circulatory system of the patient). Infusion tubing extends from the infusion port to an infusion reservoir, which is typically a plastic bag containing infusion liquid.

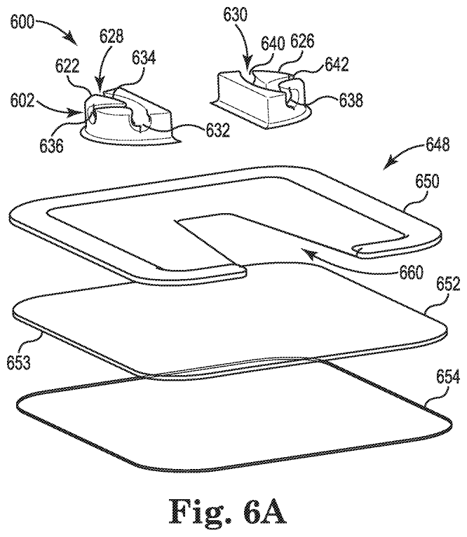

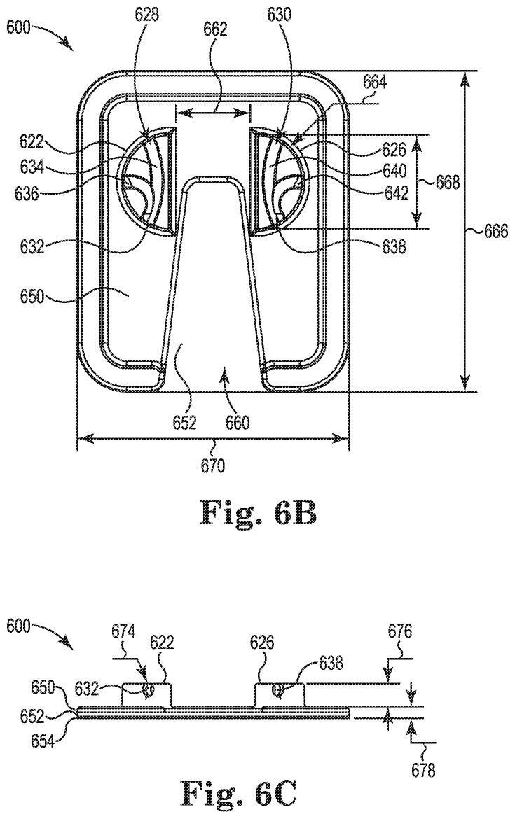

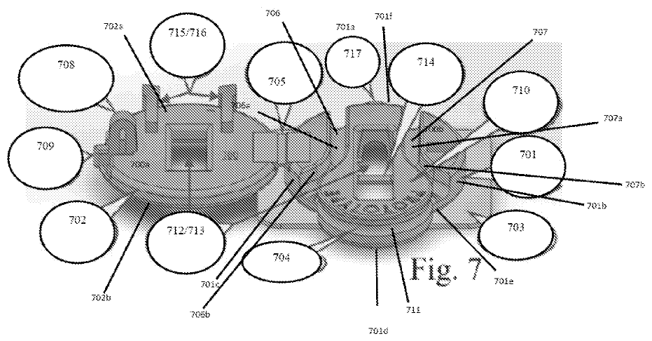

FIG. 6A is a diagram illustrating an assembly view of an infusion site retainer 600 according to another embodiment. Infusion site retainer 600 includes a base 602, and a multi-layer support structure 648. The base 602 is attachable to the support structure 648, and the support structure 648 is attachable to a patient. Support structure 648 includes a fabric layer 650, a clear plastic layer 652, and a cover layer 654. In one embodiment, the cover layer 654 is a clear plastic film. A bottom surface of the fabric layer 650 is configured to be attached to a top surface of the clear plastic layer 652. A bottom surface of the clear plastic layer 652 is configured to be attached to a top surface of the cover layer 654. In one embodiment, the bottom surface of the clear plastic layer 652 comprises an adhesive surface 653, which is initially covered by the cover layer 654. The cover layer 654 is easily removable by a user, to expose the adhesive surface 653, and allow the adhesive surface 653 to be attached to a patient's arm.

Base 602 includes a top surface opposite a bottom surface. In one embodiment, the bottom surface includes an adhesive configured to attach base 602 to the support structure 648. Base 602 includes first lateral base portion 622 and second lateral base portion 626, which are separate and distinct elements in the illustrated embodiment. In one embodiment, base 602 has dimensions that are similar to those of the base 102 described above.

First lateral base portion 622 includes a trench 628 formed in the top surface of the base portion 622. Trench 628 includes a first a first trench portion 632 that extends from the distal end of the base 602 toward the proximal end of the base 602. First trench portion 632 branches into a second trench portion 634 and a third trench portion 636. Second trench portion 634 extends from first trench portion 632 to the proximal end of the base 602. Third trench portion 636 branches laterally away from first trench portion 632 and is close to perpendicular to first trench portion 632 and second trench portion 634. Third trench portion 636 extends from first trench portion 632 to the lateral side of the base 602.

Second lateral base portion 626 includes a trench 630 formed in the top surface of the base portion 626. Trench 630 includes a first a first trench portion 638 that extends from the distal end of the base 602 toward the proximal end of the base 602. First trench portion 638 branches into a second trench portion 640 and a third trench portion 642. Second trench portion 640 extends from first trench portion 638 to the proximal end of the base 602. Third trench portion 642 branches laterally away from first trench portion 638 and is close to perpendicular to first trench portion 638 and second trench portion 640. Third trench portion 642 extends from first trench portion 638 to the lateral side of the base 602. Trench portions 632, 634, 636, 638, 640, and 642 are all curved along their length.

In one embodiment, base 602 is integrally formed from a recyclable or biodegradable plastic to include trenches 628 and 630. Base 602 according to one embodiment is formed from a thermoplastic elastomer. Trench 628 is configured to couple a first portion of the infusion tubing to base 602, and trench 630 is configured to couple a second portion of the infusion tubing to base 602. In one embodiment, trenches 628 and 630 are configured to secure the first and second portions of the tubing in a substantially immobile relationship relative to the base 602, without crimping the tubing.

For trench 628, the first portion of the infusion tubing may be secured in the first trench portion 632 and the second trench portion 634 to maintain the first portion of the tubing substantially straight, but with a small curve. Alternatively, the first portion of the infusion tubing may be secured in the first trench portion 632 and the third trench portion 636, which forms a larger curve in the infusion tubing. Similarly, for trench 630, the second portion of the infusion tubing may be secured in the first trench portion 638 and the second trench portion 640 to maintain the second portion of the tubing substantially straight, but with a small curve. Alternatively, the second portion of the infusion tubing may be secured in the first trench portion 638 and the third trench portion 642, which forms a larger curve in the infusion tubing.

In one embodiment, trenches 628 and 630 have a semicircular cross-sectional shape, and are sized to enable the infusion tubing to be pressed into the trenches 628 and 630 in a manner that captures the infusion tubing. In one embodiment, the trenches 628 and 630 are configured to clamp down upon or grab the infusion tubing, and in this manner prevent the infusion tubing from tugging on the infusion port.

The infusion site retainer 600 is attached to the patient by a healthcare worker in a suitable clean or antiseptic manner before or after the healthcare worker introduces an infusion port to the patient at the infusion site. The infusion port includes a needle that is introduced to a vein (or broadly, to the circulatory system of the patient). Infusion tubing extends from the infusion port to an infusion reservoir, which is typically a plastic bag containing infusion liquid.

FIG. 6B is a diagram illustrating a top view of the infusion site retainer 600 shown in FIG. 6A after assembly according to one embodiment. As shown in FIG. 6B, each of the base portions 622 and 626 has a semi-circular shape, with a straight side and a rounded side. The straight sides of the base portions 622 and 626 are parallel and face each other, and are separated by a distance 662, which is about 1.905 cm in one embodiment. The top surfaces of the base portions 622 and 626 have a radius of curvature 664, which is about 1.270 cm in one embodiment. The base portions 622 and 626 have a length 668, which is about 2.410 cm in one embodiment. The support structure 648 has a length 666, which is about 8.255 cm in one embodiment, and a width 670, which is about 6.985 cm in one embodiment.

An opening 660 is formed in one side of the fabric layer 650 and extends toward and beyond the center of the fabric layer 650. The width of the opening 660 becomes gradually narrower with increasing distance from the side of the fabric layer 650. Base portions 622 and 626 are positioned on opposing sides of the opening 660.

FIG. 6C is a diagram illustrating a side view of the infusion site retainer 600 shown in FIG. 6A after assembly according to one embodiment. As shown in FIG. 6C, each of the trenches 628 and 630 has a radius of curvature 674, which is about 0.191 cm in one embodiment. The base 602 has a thickness 676, which is about 0.584 cm in one embodiment. The support structure 648 has a thickness 678, which is about 0.305 cm in one embodiment.

FIG. 7 shows an infusion site retainer 700 according to another embodiment. In one embodiment, infusion site retainer 700 is provided as a stand-alone component that is attachable to the limb of the patient in any manner deemed suitable by the healthcare provider. The infusion site retainer 700 is substantially disc-shaped in a closed position, and includes a base 701, a cover 702, and a flexible base pad 703. The base 701 is attachable to a patient (not shown) through the flexible base pad 703. The infusion site retainer 700 includes a channel 704 configured to hold an infusion tube. A hinge 705 allows for the top 700a and bottom 700b to close.

The base 701 includes a first surface 701a opposite a patient surface or bottom surface 701e. The base 701 includes opposing first 701b and second lateral 701c sides, and a proximal end 701f opposite a distal end 701d. A flexible base pad 703 is attached to the bottom surface of the base 701e and is configured to be adhesively attached to the patient and conform to contours of the skin. The flexible base pad 703 may include an adhesive layer that is initially covered by a cover layer that can be easily peeled off when the retainer 700 is about to be applied to a patient. The flexible base pad 703 may be positioned on an existing bandage like a Tegaderm™ or may be positioned directly over an intravenous insertion site to protect the puncture area. The size and configuration of the flexible base pad 703 may vary (e.g., circular, rectangular, square, or other geometry), and the flexible base pad 703 may include extensions to assist in adhesion to a bandage or skin.

The base 701 includes first 706 and second trenches 707 formed in the top surface 701a of the base 701. The first trench 706 includes a first trench portion 706a that extends linearly from the proximal end of the base 701 toward the distal end of the base 701. The first trench 706 includes a second trench portion 706b that extends from the first trench portion 706a and curves laterally away from the first trench portion 706a at an angle of about forty-five degrees. The second trench portion 706b of the first trench 706 terminates at a lateral side of the base 701. The second trench 707 includes a first trench portion 707a that extends linearly from the proximal end of the base 701 toward the distal end of the base 701. The second trench 707 includes a second trench portion 707b that extends from the first trench portion 707a and curves laterally away from the first trench portion 707b at an angle of about forty-five degrees. The second trench portion 707b of the second trench 707 terminates at a lateral side of the base 701. The first trench portion 706a of the first trench 706 is substantially parallel to the first trench portion 707a of the second trench 707.

In one embodiment, the base 701 is integrally formed from a plastic to include the first 706 and second trenches 707. The first trench 706 is configured to couple a first portion of infusion tubing to the base 701, and the second trench 707 is configured to couple a second portion of the infusion tubing to the base 701. In one embodiment, the first 706 and second trenches 707 are configured to secure the first and second portions of the tubing in a substantially immobile relationship relative to the base 701, without crimping the tubing.

In one embodiment, the first 706 and second trenches 707 have a semicircular cross-sectional shape and are sized to enable the infusion tubing to be pressed into the trenches 706 and 707 in a manner that captures the infusion tubing. In one embodiment, the trenches 706 and 707 are configured to clamp down upon or grab the infusion tubing, and in this manner prevent the infusion tubing from tugging on the infusion port.

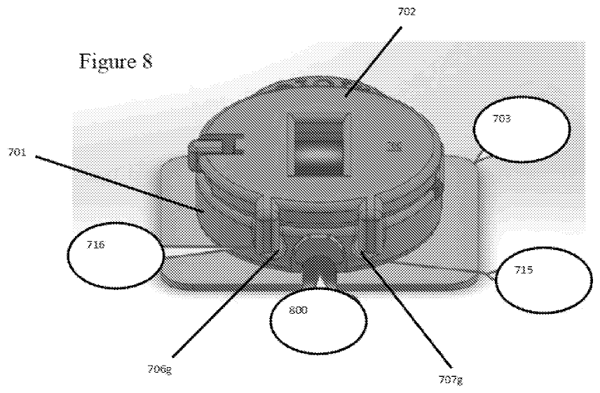

The cover 702 has a top surface 702a and a bottom surface 702b. The cover 702 is attached to the base 701 via a hinge 705 that pivotally connects a lateral side of the cover 702 to a lateral side of the base 701. The cover 702 is configured to be pivoted via the hinge 705 between an open position as shown in FIG. 7, for example, and a closed position as shown in FIG. 8, for example. The hinge 705 may be a living hinge, a butterfly hinge, a detached two-piece hinge, or another type of hinge. The cover 702 may be maintained in the closed position via a cantilever lock 708 on the lateral side of the cover 702. After closing the cover 702, the cantilever lock 708 may be snapped into place in an opening in the top surface of the base 701 to hold the cover 702 in the closed position. A pushbutton lock release 709 is coupled to the cantilever lock 708 to allow the cover 702 to be released from being locked and then opened. The cover 702 may be closed after the tubing is inserted into the trenches 706 and 707 to help secure the tubing in the base 701. In the closed position, the cover 702 laterally extends across the entire base 701 from one lateral side of the base 701 to the other lateral side of the base 701, and longitudinally extends from the proximal end of the base 701 to just short of the distal end of the base 701.

The distal portion of the base 701 includes a protuberance 710 that extends beyond the cover 702. The protuberance 710 has a semi-circular shape and includes a third trench 711 formed in a sidewall of the protuberance 710. The third trench 711 extends around the semicircular shape of the protuberance 710 of the base 701 and helps secure tubing to the protuberance 710 of the base 701, which eliminates a tubing loop that could be snagged while worn by a patient.

A first rectangular-shaped viewing window 712 is formed near a center of the cover 702. A second rectangular-shaped viewing window 713 is formed near a center of the base 701. In an example, the second viewing window 713 extends farther distally than the first viewing window 712 and extends beyond a distal end of the cover 702 when the cover 702 is in the closed position. The first and second viewing windows are substantially aligned with each other when the cover 702 is in the closed position over the base 701 and allow the infusion tip site on the patient to be viewed (e.g., by a clinician/nurse) from a top side of the retainer 700.

In some embodiments, a fourth trench is formed in the bottom surface of the base and extends from a proximal end of the viewing window formed in the base to the proximal end of the base. In some examples, the fourth trench may have a semicircular cross-sectional shape. In other examples, the fourth trench may have a rectangular or other cross-sectional shape. The fourth trench helps secure the infusion port to the proximal portion of the base. A bridge hub stabilizer is formed on a sidewall of the base at the proximal end of the base between the first and second trenches and forms a bridge over the fourth trench at the proximal end of the base. The bridge hub stabilizer 717 is configured to engage a tab on the infusion port to help secure and stabilize the infusion port. In an example, the bridge hub stabilizer 717 includes a slot sized to receive the tab on the infusion port.

A back flow cross bar 714 is formed on a bottom surface of the base 701 and extends across the second viewing window 713 of the base 701. The cross bar 714 helps to prevent backflow from a catheter when connecting or changing tubing as pressure is applied from the top of the retainer 700.

First and second channel end brackets 715 and 716 are formed at a proximal end of the cover 702. The channel end brackets 715 and 716 extend outward from a bottom surface 702a of the cover 702 and extend longitudinally from near a proximal end of the first viewing window to beyond a proximal end of the cover. When the cover 702 is in the closed position, the first and second channel end brackets 715 and 716 are respectively aligned with the first and second trenches 706 and 707 of the base 701. When the cover 702 is in the closed position, the first and second channel end brackets 715 and 716 partially extend into a proximal end portion 706e and 707e of the first and second trenches 706 and 707, respectively, to push down on the tubing and help secure the tubing in place at the proximal end of the retainer 700. Since the channel end brackets 715 and 716 extend longitudinally beyond a proximal end of the cover 702, the channel end brackets 715 and 716 help to hold the tubing flush against the skin of the patient near the proximal end of the retainer 700 to avoid movement of the tubing.

The infusion site retainer 700 may be attached to the patient by a healthcare worker in a suitable clean or antiseptic manner before or after the healthcare worker introduces an infusion port to the patient at the infusion site. The infusion port includes a needle that is introduced to a vein (or broadly, to the circulatory system of the patient). Infusion tubing extends from the infusion port to an infusion reservoir, which is typically a plastic bag containing infusion liquid.

FIG. 8 shows the infusion site retainer 700 in a closed configuration. The cover 702 is positioned over the base 701, which is connected to the flexible pad 703. The first and second channel end brackets 715 and 716 are shown extended into a proximal end portion 706g and 707g of the first and second trenches 706 and 707. In addition, there is a tunnel channel 800 placed in the base 701 and flexible pad 703.

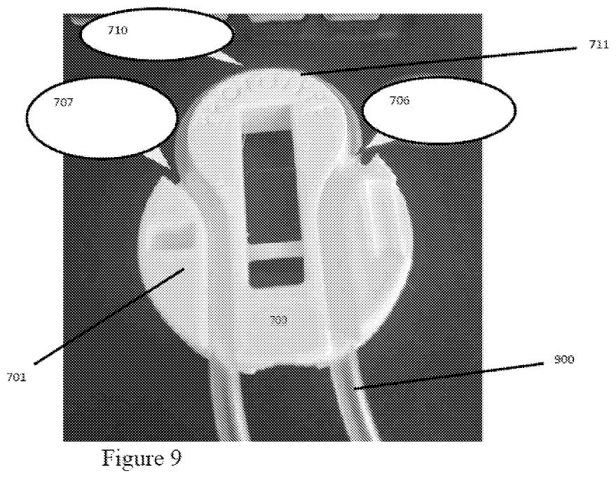

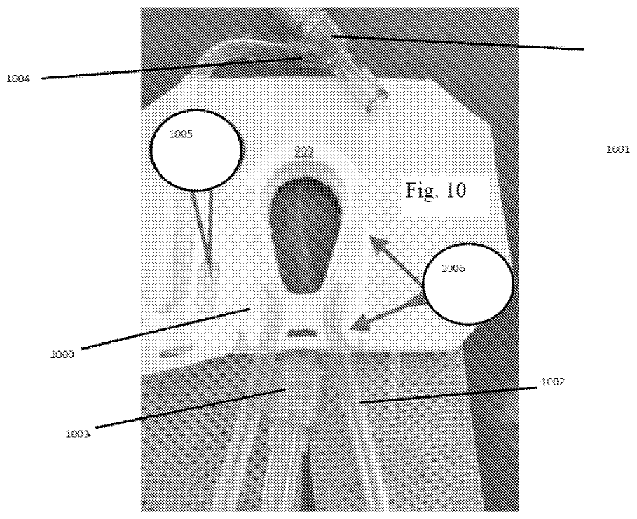

FIG. 9 shows the infusion site retainer 700 comprising the base 701 including the protuberance 710. A tube 900 is placed in the first and second trenches 706 and 707. The tube 900 wraps around the third trench 711.

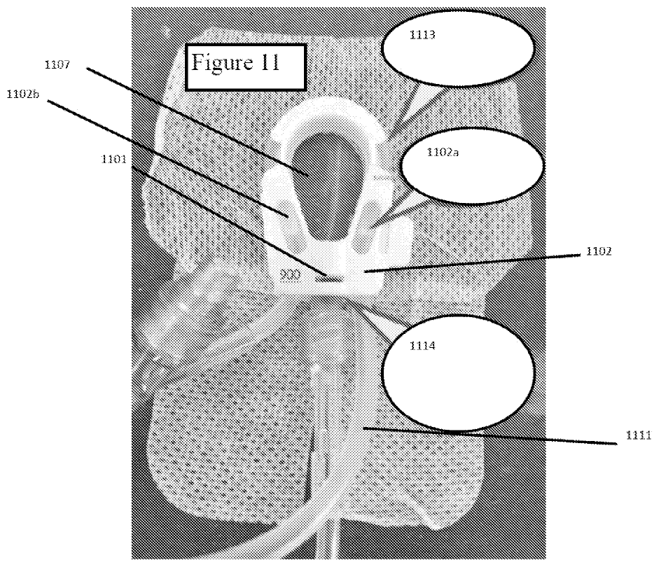

For the embodiment shown in FIG. 10, the infusion site retainer 900 has a luer 1001 and a built-in tubing channel 1002 that functions as IV tubing. The infusion site retainer 900 is also attached to a flexible pad 1005. The luer 1001 is part of the body 1000 and is flexible or stiff and attaches to the IV hub (not shown). A tubing channel 1002 for infusion made of plastic or other material is embedded in or is an intrinsic part of the device. At the anterior is a standard attachment 1003 (luer lock adapter port) for the hub of a catheter of any kind to attach to. Any luer attachment may be attached to the distal end of the device, and IV fluid, etc., may be infused through the device and into the channel built into or embedded in the device and through to the attached IV catheter. The infusion site retainer 1000 also includes a side port 1004 for medication that could be used for medications that are caustic to tubing 1002. The side port 1004 for medication may also be positioned closer to the luer 1001. The device further comprises a port window 1005 to all tubing to bulge through and channel ledges 1006 to stabilize the tubing. The device stabilizes the hub.

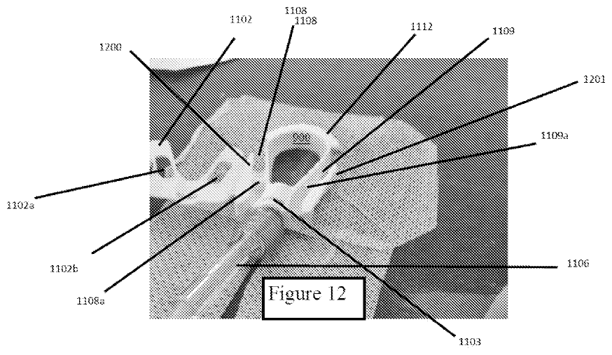

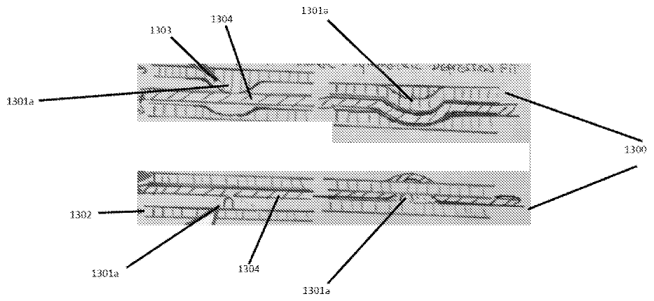

As shown in FIG. 11 and FIG. 12, the device 900 includes a slit 1101 near the proximal end of the cover 1102 and a corresponding slit 1103 in the body of the device. A push tab that is part of the IV catheter 1106 comes through the slits 1101 and 1103. The device 900 includes a lightbulb shaped viewing window 1107 that extends through the cover 1102 and the body. The device 900 further includes an opening 1114 to allow tubing to enter the device 900. The cover 1102 includes openings 1102a and 1102b near lateral ends of the cover 1102. The openings 1102a and 1102b are aligned with channels 1108 and 1109, as well as anterior channel 1113 in the base 1110 of the devices 900 that hold the tubing 1111. Each of the channels 1108 and 1109 includes an elevation tab 1108a and 1109a that extends laterally across the channel 1108 and 1109 and lifts the tubing 1111 upward toward the openings 1102a and 1102b in the cover 1102. The elevation tabs 1108a and 1109a function as a vertical strain relief feature. In other examples, the vertical strain relief may be more of a mound or a hump rather than a narrower tab. The cover 1102 extends from a proximal end of the device 900 to just beyond halfway to a distal end of the device 900. The flange 1112 at the distal end of the device 900 is a continuous flange that extends outward in an arc shape from a distal arc-shaped portion of the device 900. In other examples, the flange 1112 may be separated into separate flange portions. The flange 1112 helps to hold tubing 1111 in place in a channel of the device 1100. FIG. 12 show the device 900 with the cover opened up. The two channels 1108 and 1109 in the body that extend from the proximal end toward the distal end include ledges 1200 and 1201 that extend partially across the channel to help hold the tubing in place inside the channel 1108 and 1109 when the cover 1102 is opened up.

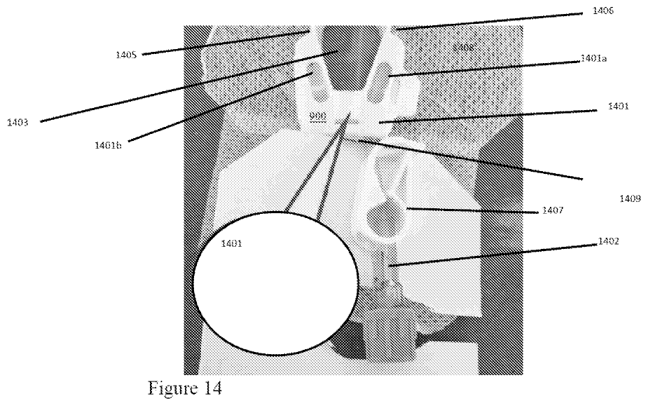

FIG. 13 shows the infusion site retainer 1300 may include features to provide channel strain relief 1301 for tubing, including channel reliefs 1301 that rise up 1301a from the base 1302 or descend down 1301b from the cover 1303. The cover 1303 closes over the tubing 1304 and pushes slightly into the tubing 1304 to secure the tubing in place. The strain relief includes convex and concave methods.

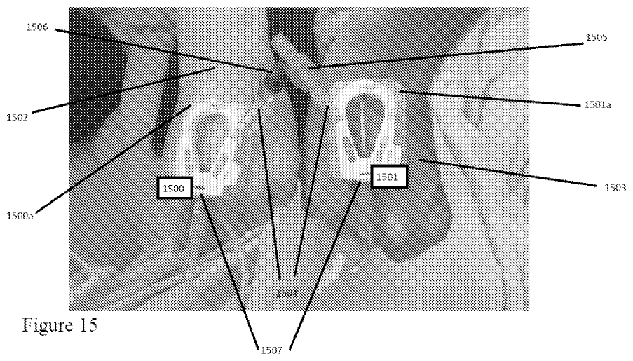

FIG. 14 show that the device 900 comprising cover 1401 and tube 1402. The device 900 includes a lightbulb shaped viewing window 1403 that extends through the cover 1401 and the base. The cover 1401 includes openings 1401a and 1401b near lateral ends of the cover 1401. The openings 1401a and 1401b are aligned with channels 1405 and 1406 in the base of the devices 900 that hold the tubing 1402. The device 900 further comprises a flexible pad 1408 that is adherent to a patient's skin. The tube 1402 comprises a clamp 1407 to regulate fluid flow. The device 900 further contains a bridge hub 1409 to accommodate a push off tab of an IV catheter. The bridge hub 1409 deters rotation, advance, or pulling out of a catheter.

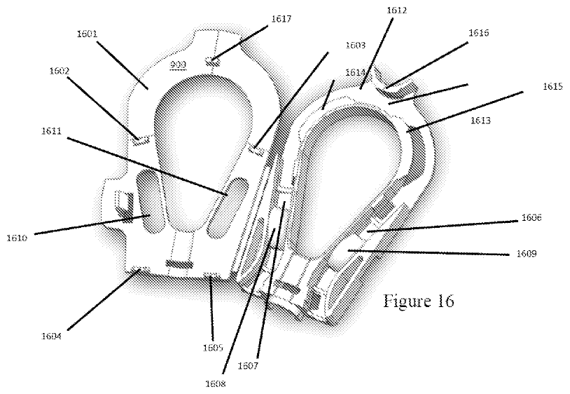

FIG. 15 shows two devices 1500 and 1501 connected to patient hands 1502 and 1503. These devices are similar to the device shown in FIG. 11. FIG. 15 further shows tubing 1504 extending from port 1505 and connecting to luer lock 1506. Devices 1500 and 1501 further comprise bridge hub 1507 to accommodate a push off tab of an IV catheter tubing 1504. Devices 1500 and 1501 also comprise flexible pads 1500a and 1501a that adhere to patient hands 1502 and 1503.

FIG. 16 shows the infusion site retainer 900 with four channel brackets 1602, 1603, 1604, and 1605 that extend from the cover 1601. The first and second channel brackets 1602 and 1603 are positioned at a proximal end of the cover 1601 that are aligned over proximal ends of first and second channels 1606 and 1607, respectively, of the body 900, when the cover 1601 is closed. The first and second channel brackets 1602 and 1603 push the tubing into the first and second channels 1606 and 1607, respectively, when the cover 1601 is closed, to help secure the tubing at the proximal end of the channels 1606 and 1607. The third and fourth channel brackets 1604 and 1605 positioned distally from the proximal end of the cover 1601 that are aligned over distal ends of the first and second channels 1606 and 1607, respectively, of the body 900, when the cover 1601 is closed. The third and fourth channel brackets 1604 and 1605 push the tubing into the first and second channels 1606 and 1607, respectively, when the cover 1601 is closed, to help secure the tubing at the distal end of the channels 1606 and 1607. The infusion site retainer 900 also includes first and second vertical strain relief features 1608 and 1609 formed in the first and second channels 1606 and 1607, respectively, which push the tubing upward toward the cover 1601. The cover 1601 includes first and second oval shaped openings 1610 and 1611 that are aligned over the first and second vertical strain relief features 1608 and 1609, respectively, when the cover 1601 is closed. The first and second openings 1610 and 1611 allow the tubing to protrude through the cover 1601 as it is forced upward by the vertical strain relief features 1608 and 1609, which helps immobilize the tubing. The body 900 includes a curved distal end 1612 with features to secure tubing at the distal end, including a curved channel 1613 to hold the tubing, flanges 1614 and 1615 extending over the channels, and a bumper guide 1616 to direct the tubing anterior. The cover 1601 includes a tab 1617 that extends partially into the curved channel 1613 when the cover 1601 is closed to help secure the tubing at the distal end of the device 900.

The infusion site retainers disclosed herein can set and anchor infusion tubing securely and allow a healthcare worker to make loops and tubing adjustments, including changing the direction of the tubing, without having to use tape and without having to remove the retainer. In some examples, infusion site retainers may be used to organize and secure various types of catheters or tubing, including PICC Line, Portacath, Central Line, Urinary Catheter, Suprapubic Catheter, and Drain Catheter. Various modifications may be made to the trench sizes, and other modifications may be made to the configuration and dimensions of the site retainers to facilitate these applications.

One embodiment is directed to an infusion site retainer configured to maintain tubing coupled to an infusion port. The infusion site retainer includes a base configured to be secured to a patient and comprising a patient surface opposite a first surface, and at least one trench formed in the base to couple the tubing to the base.

The at least one trench according to one embodiment comprises a first trench that includes a first trench portion that extends from a distal end of the base toward a proximal end of the base, and the first trench portion branches into a second trench portion and a third trench portion. The second trench portion is substantially parallel to the first trench portion, and the second trench portion extends from the first trench portion to the proximal end of the base. The third trench portion branches laterally away from first trench portion and is substantially perpendicular to the first trench portion and the second trench portion and extends from the first trench portion to a lateral side of the base. In one embodiment, the at least one trench comprises a second trench configured in a same manner as the first trench.

The infusion site retainer according to one embodiment further includes a plurality of locking features that facilitate locking a portion of the tubing in the at least one trench, wherein each of the locking features extends over and covers a portion of the at least one trench.

In one embodiment, the base includes a first lateral base portion, a central base portion, and a second lateral base portion, and wherein the central base portion is positioned between the first lateral base portion and the second lateral base portion, and wherein the first lateral base portion and the second lateral base portion have a larger thickness than the central base portion.

In one embodiment, the infusion site retainer further includes a cover configured to cover the first surface of the base. The cover according to one embodiment includes a first cover portion, a second cover portion, and a semicylindrical protrusion positioned between the first cover portion and the second cover portion. In one embodiment, the cover includes a hinge attachment feature that pivotally connects the cover to the base, and the cover is configured to be pivoted via the hinge attachment feature between an open position and a closed position. In one embodiment, the infusion site retainer further includes a clip configured to maintain the cover in the closed position.

In one embodiment, the infusion site retainer further includes at least one trench formed in a bottom surface of the cover. The at least one trench formed in the bottom surface of the cover is aligned with and lies directly over the at least one trench formed in the base when the cover is in a closed position.

In one embodiment, the base includes a first lateral base portion and a second lateral base portion, which are separate and distinct elements, and the infusion site retainer further includes a support structure, wherein the first lateral base portion and the second lateral base portion are positioned on the support structure. The support structure according to one embodiment includes a fabric layer, a clear plastic layer, and a cover layer. A bottom surface of the clear plastic layer comprises an adhesive surface that is covered by the cover layer, and the cover layer is removable by a user to expose the adhesive surface for attachment to a patient. In one embodiment, an opening is formed in one side of the fabric layer and extends toward and beyond a center of the fabric layer.

In one embodiment, each of the first lateral base portion and the second lateral base portion has a semi-circular shape, with a straight side and a rounded side. The straight sides of the first lateral base portion and the second lateral base portion are parallel and face each other.

Another embodiment is directed to a method of securing tubing coupled to an infusion port. The method includes attaching a base to the patient and retaining the tubing in at least one trench formed in the base.

Another embodiment is directed to an infusion site retainer configured to maintain tubing coupled to an infusion port. The infusion site retainer includes a base configured to be secured to a patient and comprising a patient surface opposite a first surface and comprising a sidewall surface substantially perpendicular to the patient surface and the first surface. The infusion site retainer includes at least one trench formed in the first surface of the base to secure the tubing to the base, and at least one trench formed in the sidewall surface of the base to secure the tubing to the base.

The at least one trench formed in the first surface of the base may include a first trench, wherein the first trench includes a first trench portion that extends linearly from a proximal end of the base toward a distal end of the base, and a second trench portion that extends from the first trench portion and curves laterally away from the first trench portion, wherein the second trench portion terminates at a first lateral side of the base. The at least one trench formed in the first surface of the base may include a second trench, wherein the second trench includes a first trench portion that extends linearly from the proximal end of the base toward the distal end of the base, and a second trench portion that extends from the first trench portion and curves laterally away from the first trench portion, wherein the second trench portion of the second trench terminates at a second lateral side of the base. The infusion site retainer may further include a trench formed in the patient surface of the base to secure the infusion port in place. The infusion site retainer may include a stabilizer element formed at a proximal end of the base adjacent to the trench formed in the patient surface, wherein the stabilizer element is configured to engage a tab on the infusion port to help secure the infusion port in place.

The infusion site retainer may further include a cover attached to the base via a hinge, wherein the cover is configured to be pivoted via the hinge between an open position in which the first surface of the base is exposed and a closed position to cover the first surface of the base. The cover may include a locking mechanism to lock the cover to the base in the closed position, and a push button to unlock the locking mechanism. The base may include a viewing window, wherein the cover includes a viewing window, and wherein the viewing window of the base is aligned with the viewing window of the cover when the cover is in the closed position to allow viewing through the infusion site retainer. The base may include a cross bar formed on a bottom surface of the base, and wherein the cross bar extends across the viewing window of the base. The infusion site retainer may further include at least one bracket extending outward from a bottom surface of the cover and extending longitudinally beyond a proximal end of the cover to hold the tubing flush against skin of a patient when the cover is in the closed position. The hinge may include a cylindrical shaft and a semicircular-shaped clip configured to be snapped into place over the cylindrical shaft to pivotally connect the base and the cover.

A distal portion of the base may include a protuberance that has a semicircular shape, and wherein the at least one trench formed in the sidewall surface of the base is formed in a sidewall surface of the protuberance. The infusion site retainer may further include a flexible base pad attached to the patient surface of the base, wherein the flexible base pad includes an adhesive layer covered by a cover layer, and wherein the cover layer is removable to allow the flexible base pad to be adhesively attached to a patient. The infusion site retainer may be substantially disc shaped. The infusion site retainer may further include at least one vertical strain relief feature to help immobilize the tubing. The at least one vertical strain relief feature may be formed in the at least one trench. The infusion site retainer may further include at least one arc strain relief feature to help immobilize the tubing and help prevent kinking of the tubing at an arc in an apex area of the body. The cover may further include a luer guard extending from a base end portion of the cover, wherein the luer guard is configured to cover and secure a luer/IV hub connection when the cover is in the closed position.

Another embodiment is directed to a method of securing tubing coupled to an infusion port. The method includes attaching a base to the patient. The method includes retaining the tubing in at least one trench formed in a first surface of the base. The method includes covering the first surface of the base with a cover, wherein the base and the cover each include a viewing window to allow viewing through the base and the cover.

Although the present invention has been described with reference to preferred embodiments, workers skilled in the art will recognize that changes can be made in form and detail without departing from the spirit and scope of the present invention.

What is claimed is:

1. An infusion site retainer configured to maintain tubing coupled to an infusion port, the infusion site retainer comprising:
a base configured to be secured to a patient and comprising a patient surface opposite a first surface and comprising a sidewall surface substantially perpendicular to the patient surface and the first surface; at least one trench formed in the first surface of the base to secure the tubing to the base; and at least one trench formed in the sidewall surface of the base to secure the tubing to the base.

2. The infusion site retainer of claim 1, wherein the at least one trench formed in the first surface of the base includes a first trench, wherein the first trench includes a first trench portion that extends linearly from a proximal end of the base toward a distal end of the base, and a second trench portion that extends from the first trench portion and curves laterally away from the first trench portion, wherein the second trench portion terminates at a first lateral side of the base.

3. The infusion site retainer of claim 2, wherein the at least one trench formed in the first surface of the base includes a second trench, wherein the second trench includes a first trench portion that extends linearly from the proximal end of the base toward the distal end of the base, and a second trench portion that extends from the first trench portion and curves laterally away from the first trench portion, wherein the second trench portion of the second trench terminates at a second lateral side of the base.

4. The infusion site retainer of claim 1, and further comprising a trench formed in the patient surface of the base to secure the infusion port in place.

5. The infusion site retainer of claim 4, and further comprising a stabilizer element formed at a proximal end of the base adjacent to the trench formed in the patient surface, wherein the stabilizer element is configured to engage a tab on the infusion port to help secure the infusion port in place.

6. The infusion site retainer of claim 1, and further comprising a cover attached to the base via a hinge, wherein the cover is configured to be pivoted via the hinge between an open position in which the first surface of the base is exposed and a closed position to cover the first surface of the base.

7. The infusion site retainer of claim 6, wherein the cover includes a locking mechanism to lock the cover to the base in the closed position, and a push button to unlock the locking mechanism.

8. The infusion site retainer of claim 6, wherein the base includes a viewing window, wherein the cover includes a viewing window, and wherein the viewing window of the base is aligned with the viewing window of the cover when the cover is in the closed position to allow viewing through the infusion site retainer.

9. The infusion site retainer of claim 8, wherein the base includes a cross bar formed on a bottom surface of the base, and wherein the cross bar extends across the viewing window of the base.

10. The infusion site retainer of claim 6, and further comprising at least one bracket extending outward from a bottom surface of the cover and extending longitudinally beyond a proximal end of the cover to hold the tubing flush against skin of a patient when the cover is in the closed position.

11. The infusion site retainer of claim 6, wherein the hinge includes a cylindrical shaft and a semicircular-shaped clip configured to be snapped into place over the cylindrical shaft to pivotally connect the base and the cover.

12. The infusion site retainer of claim 1, wherein a distal portion of the base includes a protuberance that has a semi-circular shape, and wherein the at least one trench formed in the sidewall surface of the base is formed in a sidewall surface of the protuberance.

13. The infusion site retainer of claim 1, and further comprising a flexible base pad attached to the patient surface of the base, wherein the flexible base pad includes an adhesive layer covered by a cover layer, and wherein the cover layer is removable to allow the flexible base pad to be adhesively attached to a patient.

14. The infusion site retainer of claim 1, wherein the infusion site retainer is substantially disc-shaped.

15. The infusion site retainer of claim 1, and further including at least one vertical strain relief feature to help immobilize the tubing.

16. The infusion site retainer of claim 15, wherein the at least one vertical strain relief feature is formed in the at least one trench.

17. The infusion site retainer of claim 1, and further including at least one arc strain relief feature to help immobilize the tubing and help prevent kinking of the tubing at an arc in an apex area of the body.

18. The infusion site retainer of claim 6, wherein the cover further includes a luer guard extending from a base end portion of the cover, wherein the luer guard is configured to cover and secure a luer/IV hub connection when the cover is in the closed position.

19. A method of securing tubing coupled to an infusion port, the method comprising:
attaching a base to the patient;
retaining the tubing in at least one trench formed in a first surface of the base; and
covering the first surface of the base with a cover, wherein the base and the cover each include a viewing window to allow viewing through the base and the cover.
